SEQUENCE LISTING (11 sequences)

SEQ ID NO: 1 - DNA, 61 bp, Artificial Sequence
  fragments of human genome
acatgaaaaactatgtgcaaagaagtggagntgcagaagagctttgaatttggagaactgt

SEQ ID NO: 2 - DNA, 61 bp, Artificial Sequence
  fragments of human genome
aatcaagtgacttatatttagatctgcaaanaaaacatttccttctgctgagtacatctag

SEQ ID NO: 3 - DNA, 61 bp, Artificial Sequence
  fragments of human genome
ccttatttggaaaatggatataatcaaaatnatggaagaaagtttgtacagggtaaatcta

SEQ ID NO: 4 - DNA, 61 bp, Artificial Sequence
  fragments of human genome
ctgtctttatcacactccatgatcccagctnctaaaatccacactgagctctgcttaccag

SEQ ID NO: 5 - DNA, 61 bp, Artificial Sequence
  fragments of human genome
cctgaacgacaactcccgggatatgactgangtgatcagcagcctggaaaatgccaactat

SEQ ID NO: 6 - DNA, 61 bp, Artificial Sequence
  fragments of human genome
aggatggtgaggagcccgccagaggcccgtnttgggaacctggacacagtgcccctcactt

SEQ ID NO: 7 - DNA, 61 bp, Artificial Sequence
  fragments of human genome
acgccatggggagcaatctcagcccccaacnctgcctgatgccctttatcttgggcctctt

SEQ ID NO: 8 - DNA, 62 bp, Artificial Sequence
  fragments of human genome
gtgtgaccaccactccatggtctttggcccngccccaggggatcctgctctctggagggggt

SEQ ID NO: 9 - DNA, 61 bp, Artificial Sequence
  fragments of human genome
aggtggcctgcacggtcacgaacttgttgcnccccttttctgacttgaactccctgttggc

SEQ ID NO: 10 - DNA, 61 bp, Artificial Sequence
  fragments of human genome
ctgccagctccgcggacgctgccttcgtccngccgcagaggccccgcggtcagggtcccgc

SEQ ID NO: 11 - DNA, 61 bp, Artificial Sequence
  fragments of human genome
cctgcagtccctgcccggcccagtccgagcntcccgggcgggcccccagtccggcgatttg

(19) United States
(12) Patent Application Publication
(10) Pub. No.: US 2012/0276084 A1
(43) Pub. Date: Nov. 1, 2012

(54) Predicting Risk of Age-Related Macular Degeneration

(75) Inventors: Debra A. Schaumberg, Cambridge, MA (US); Daniel Chasman, Cambridge, MA (US)

(73) Assignee: THE BRIGHAM AND WOMEN'S HOSPITAL, INC., Boston, MA (US)

(21) Appl. No.: 13/404,648

(22) Filed: Feb. 24, 2012

Related U.S. Application Data
(60) Provisional application No. 61/446,823, filed on Feb. 25, 2011.

(51) Int. Cl. C12Q 1/68; A61K 39/395; A61P 27/02; C40B 30/04

(52) U.S. Cl. 424/133.1; 435/6.11; 435/6.12; 506/9

(57) ABSTRACT

This invention relates to methods for predicting risk of developing age-related macular degeneration (AMD), based on detecting the presence of certain genetic variants on chromosome 16 that are associated with increased incidence of AMD.

### CLAIM OF PRIORITY

This application claims the benefit of U.S. Provisional Patent Application Ser. No. 61/446,823, filed on Feb. 25, 2011, the entire contents of which are hereby incorporated by reference.

### FEDERALLY SPONSORED RESEARCH OR DEVELOPMENT

This invention was made with Government support under Grant No. EY017362, IIL043851, HL080467, and CA047988 awarded by the National Institutes of Health. The Government has certain rights in the invention.

### TECHNICAL FIELD

This invention relates to methods for predicting risk of developing age-related macular degeneration (AMD), based on detecting the presence of certain genetic variants on chromosome 16 that are associated with increased incidence of AMD.

### BACKGROUND

Age-related macular degeneration (AMD) is an aging-associated disease characterized by progressive degeneration of photoreceptors and/or retinal pigment epithelium (RPE) in the central part of the human retina (called the macula), which eventually leads to the loss of central vision (Swaroop et al., Human Molecular Genetics, 2007; 16(2):R174-R182). AMD is a multifactorial disease arising from the interplay between genetically determined risk and exposure to environmental/lifestyle risk factors such as cigarette smoking, dietary factors, and obesity. AMD is a substantial public health concern as it affects central vision, and the ability to read, write, drive, watch TV, and recognize faces. AMD is also associated with other co-morbidities including depression and distress (Klein et al., Ophthalmology. 1992; 99:933-943; Fisher et al., Hum Mol Genet. 2005; 14:2257-2264). Approximately 8% of Americans in their fifties have AMD, whereas 30% of persons ≧75 years of age are affected (Klein et al., Ophthalmology. 1992; 99:933-943). The prevalence of late AMD increases from 0.1% to 7.1% respectively in these age groups.

Important discoveries over the past few years identified a number of strong genetic susceptibility factors for AMD including variations in complement factor H (CFH), complement component 3 (C3), ARMS2/HTRA1, and complement factor B (CFB) (Swaroop et al., Annu Rev Genomics Hum Genet. 2009; 10:19-43). However, knowledge of AMD, how it develops, and how it might best be prevented remains incomplete and the ability to accurately predict who will develop AMD is not strong. A comprehensive understanding of the genetic factors and biological mechanisms involved in AMD pathogenesis is needed to identify those at risk and prevent vision loss in the future.

In that regard, systematic analysis of prior genome-wide linkage studies identified at least five regions of the genome that were significantly associated with AMD (Fisher et al., Hum Mol Genet. 2005; 14:2257-2264). Subsequent studies have shown clear evidence for association with the CFH gene on 1q, and with ARMS2/HTRA1 on 10q (see, e.g., Schaumberg et al., Arch Ophthalmol. 2007; 125:55-62; Swaroop et al., Annu Rev Genomics Hum Genet. 2009; 10:19-43), but no associations have yet been discovered for the other significant linkage regions on chromosomes 2p, 3p, and 16q. These regions might still harbor important genetic variants that affect AMD susceptibility.

### SUMMARY

The present invention is based, at least in part, on the discovery of two SNPs on chromosome 16 that were significantly associated with AMD when adjusted for the number of SNPs evaluated in this region: rs4887603 (OR=3.39, corrected P=0.02), and rs1858993 (OR=1.71, corrected P=0.01). SNP rs1858993 on chromosome 16 was also significantly associated with neovascular AMD (OR=3.25, corrected P=0.007), and this association remained significant after adjusting for all SNPs in all evaluated regions.

Thus, in a first aspect, the invention provides methods for predicting risk of developing age-related macular degeneration (AMD) in a human subject. The methods include detecting one or both of:

(i) the presence of a “G” allele at polymorphism rs1858993, and/or

(ii) the presence of a “T” allele at polymorphism rs4887603.

The presence of one or both of a “G” allele at rs1858993, or a “T” allele at rs4887603 indicates that the subject has an increased risk of developing AMD.

In a further aspect, the invention provides methods for selecting a treatment regimen for a subject. The methods include detecting one or both of:

(i) the presence of an “G” allele at polymorphism rs1858993, and/or

(ii) the presence of a “T” allele at polymorphism rs4887603.

The presence of one or both of a “G” allele at rs1858993, or a “T” allele at rs4887603 indicates that the subject has an increased risk of developing age-related macular degeneration (AMD); and selecting a regimen comprising a treatment for AMD for a subject who has an increased risk of developing AMD.

In some embodiments, the methods further include detecting the presence of one or more additional genetic variants associated with altered risk of AMD, e.g., as listed in FIG. 3, e.g., one or more of a Y402H variant of complement factor H(CFH), identified by the presence of a “C” allele at rs1061170; an A69S variant of ARMS2, identified by the presence of a “G” allele at rs10490924; an E318D variant of complement component 2 (C2), identified by the presence of a “C” allele at rs9332739; an L9H variant of complement factor B (CFB), identified by the presence of an “A” allele at rs4151667; an R32Q variant of complement factor B (CFB), identified by the presence of a “G” allele at rs641153; an R80G variant of complement component 3 (C3), identified by the presence of a “G” allele at rs2230199; an “A” allele at rs11200638; a “C” allele at rs2672598; and a “T” allele at rs547154.

In some embodiments of the methods described herein, the AMD is neovascular AMD.

In some embodiments of the methods described herein, determining the identity of an allele comprises obtaining a sample comprising DNA from the subject, and determining identity of the nucleotide at the polymorphism.

In some embodiments of the methods described herein, determining the identity of the nucleotide comprises contacting the sample with a probe specific for a selected allele of the polymorphism, and detecting the formation of complexes between the probe and the selected allele of the polymorphism, wherein the formation of complexes between the probe and the test marker indicates the presence of the selected allele in the sample.

In some embodiments of the methods described herein, determining the identity of an allele comprises determining the identity of the nucleotide at position 31 of one of SEQ ID NOs: 1 or 2.

In some embodiments of the methods described herein, the subject is a patient having or suspected of having AMD.

In some embodiments of the methods described herein, the subject has one or more risk factors associated with AMD.

In some embodiments of the methods described herein, the methods further include selecting the subject based on the presence of one or more risk factors associated with AMD.

In some embodiments of the methods described herein, the risk factors associated with AMD include one or more of: family history of AMD, age, sex, smoking history, obesity, body mass index (BMI), waist circumference, waist-hip ratio, weight change since age 20, dietary fat intake, linoleic acid intake, and elevated cholesterol levels.

In some embodiments of the methods described herein, the subject has exhibited or exhibits symptoms of AMD.

In some embodiments, the methods described herein include selecting or excluding a subject for enrollment in a clinical trial based on the identity of the allele.

In some embodiments, the methods described herein include stratifying a subject population for analysis of a clinical trial based on the identity of the allele in the subjects.

In some embodiments, the methods described herein include monitoring the subject for the development of AMD.

In some embodiments, the methods described herein include selecting a treatment for AMD if the subject has an increased risk of developing AMD. In some embodiments, the methods described herein include administering the selected treatment to the subject.

As used herein, “age-related macular degeneration” or “AMD” includes early, intermediate, and advanced AMD. “Advanced AMD” includes both dry AMD and wet AMD (wet AMD is also referred to as neovascular AMD). An “AMD biomarker” is a marker associated with development of AMD, or progression to advanced AMD, in a subject, as described herein.

As used herein, a “subject” is a mammal, e.g., human, canine, feline, ovine, primate, equine, porcine, caprine, camelid, avian, bovine, and murine organisms. Typically, the subject is a human. As used herein, “apparently healthy” means that a subject does not have clinical signs of AMD. In other words, such subjects, if examined by a medical professional, would be characterized as generally free of symptoms or signs of AMD.

As used herein, a “risk factor” means a risk factor that is known to be associated with an increased risk of development or progression of AMD.

As used herein, the term “stratification” refers to the creation of a distinction between subjects on the basis of a characteristic or characteristics of the subjects. Generally, in the context of clinical trials, the distinction is used to classify or separate responses or effects in different sets of patients distinguished according to the stratification parameters. In some embodiments, stratification includes distinction of subject groups based on the presence or absence of particular alleles or genotypes described herein. The stratification can be performed, e.g., in the course of analysis, or can be used in creation of distinct groups or in other ways.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety, including the entirety of U.S. Ser. No. 61/304,827, filed Feb. 16, 2010. In case of conflict, the present specification, including definitions, will control.

Other features and advantages of the invention will be apparent from the following detailed description and figures, and from the claims.

### DESCRIPTION OF DRAWINGS

FIG. 1 is table showing the association of SNPs in three positional candidate regions with age-related macular degeneration in the Women's Genome Health Study (WGHS). * The Table summarizes the top SNP (lowest P-value) for each AMD endpoint in each region based on P-values derived from chi-square tests of association as well as based on a logistic regression model adjusted for age. SNPs have minimum p-value for association with AMD-type as indicated in the Women's Genome Health Study across each of the linkage regions (“Region”). “Uncorrected P-value” refers to this minimum p-value. “Sidak-corrected P-value” refers to the p-value adjusted by the Sidak procedure for all SNPs within the linkage region (“Number of SNPs tested in region”).

† Allele 1=the minor allele for each SNP (i.e., the allele tested for association with AMD); Allele 2=the more common allele for each SNP

‡ MAF=minor allele frequency

§ CHR=chromosome

¶ AMD 20/30=all confirmed cases of AMD associated with vision loss of 20/30 or worse due to AMD; All AMD=all confirmed cases of AMD associated with or without vision loss; Neovascular=all confirmed cases of AMD with vision loss of 20/30 or worse with signs of neovascular AMD in at least one eye.

£ ASSOC=the allele frequency chi-square test of association; LOGISTIC=the test of the coefficient coding for additive effects of the genotype from an age-adjusted logistic regression model

[symbol] Odds ratio estimates are based on an additive genetic model and therefore represent the odds ratio for those with one versus no copies of the risk alleles. Square the odds ratio to calculate the odds ratio for those with two versus no copies of the risk allele.

FIG. 2 is a table showing odds ratios and P-values* in three confirmation cohorts for association with AMD of the three top SNPs identified by regional screening in the WGHS.

*Odds ratio and p-values calculated from conditional logistic regression models adjusted for age, sex, and cigarette smoking Odds ratio estimates are based on an additive genetic model and therefore represent the odds ratio for those with one versus no copies of the risk alleles. Square the odds ratio to calculate the odds ratio for those with two versus no copies of the risk allele.

‡ Odds ratio estimate for rs4887603 in the NHS/HPFS is based on dominant genetic model comparing subjects with either 1 or 2 copies of the risk allele versus no copies. There was only 1 subject in this population with 2 copies of the risk allele.

FIG. 3 is a list of the SNPs described herein, with the reference sequences and alleles associated with increased risk, as well as additional genetic variants associated with altered risk of AMD. All flanking sequences listed with respect to the “+” strand (NCBI B36/hg18).

### DETAILED DESCRIPTION

Genetic studies have identified strong associations between several common variants and risk of AMD, the strongest known associations being with complement pathway genes including complement factor H on chromosome 1q, as well as with ARMS2/HTRA1 on chromosome 10q (Swaroop et al., Annu Rev Genomics Hum Genet. 2009; 10:19-43). These regions were initially identified by linkage studies that also identified regions on chromosomes 2, 3, and 16 that might harbor additional AMD-associated variants (Fisher et al., Ophthalmology. 1992; 99:933-943).

The present study searched these genomic regions for AMD-associated common variants using data from 23,294 participants in the Women's Genome Health Study (WGHS). These analyses confirmed strong AMD associations on chromosomes 1q32 and 10q26, but failed to provide strong evidence of further AMD susceptibility loci within 2p25.1-2p16.2, 3p25.3-3p14.1, and 16p13-16q23.1.

As described herein, there was an increased risk of AMD associated with the “G” allele for rs1858993 on chromosome 16, which is intergenic between SHISA9 and the DNA repair gene ERCC4. Data from the WGHS indicates that the “G” allele of rs1858993 is very common in the US population, with a minor allele frequency of 0.27. Initial estimates from incident cases of neovascular AMD in the WGHS identified a >3-fold increased risk for carriers of 1 copy of the “G” allele, and >10-fold increased for individuals who carried 2 copies.

Although this finding was not replicated in subsequent analysis of three independent study populations, in which non-significant odds ratios were observed for neovascular AMD ranging from 1.02-1.33 for 1 copy of the “G” allele, and 1.04-1.77 for 2 copies of the “G” allele, a significant interaction indicating an increased risk of AMD for the “G” allele of rs1858993 was observed among those homozygous for the CFH Y402H variant in both the Greek case-control study as well as the population of extremely discordant sibling pairs. In addition, the odds ratios in the three replication cohorts were of more modest magnitude compared with the initial finding in the WGHS, suggesting that larger studies could confirm or fully refute an independent association. For example, a recent very large-scale study of up to 10,049 AMD cases and 7,148 controls (combining data from several populations) was able to identify novel loci with only a modest impact on AMD (e.g., odds ratios as low as 1.14) (Chen et al., Proc Natl Acad Sci USA. 2010; 107:7401-7406). In pooled analyses from the three replication cohorts an odds ratio for the “G” allele of rs1858993 of 1.14 was observed comparing neovascular AMD versus normal subjects but this was not statistically significant (P=0.10).

Taken together, the present findings suggest that rs1858993 or other variants in the region of rs1858993 influence AMD, either alone or in concert with CFH or other AMD-associated genes.

### Methods of Determining the Identity of an Allele or Obtaining a Genotype

The methods described herein include determining the identity, presence or absence of alleles or genotypes associated with diagnosis or risk of developing AMD. In some embodiments, an association with AMD is determined by the statistical likelihood of the presence of an allele or genotype in an individual with AMD, e.g., an unrelated individual or a first or second-degree relation of the subject, and optionally the statistical likelihood of the absence of the same allele or genotype in an unaffected reference individual, e.g., an unrelated individual or a first or second-degree relation of the subject. Thus the methods can include obtaining and analyzing a sample from a suitable reference individual.

As used herein, an “allele” is one of a pair or series of genetic variants of a polymorphism at a specific genomic location.

As used herein, “genotype” refers to the diploid combination of alleles for a given genetic polymorphism. A homozygous subject carries two copies of the same allele and a heterozygous subject carries two different alleles.

As used herein, “genetic model” refers to the manner in which an allele influences risk or differential diagnosis. In a “dominant model” the allele impacts the clinical state to the same extent whether present in one copy or two copies, i.e., whether homozygous or heterozygous. In a “recessive model” the allele impacts the clinical state only when homozygous. In an “additive model” the allele impacts the clinical state in proportion to the number of copies present, i.e. the homozygous state has twice the impact of the heterozygous state.

As used herein, a “haplotype” is one or a set of signature genetic changes (polymorphisms) that are normally grouped closely together on the DNA strand, and are usually inherited as a group; the polymorphisms are also referred to herein as “markers.” A “haplotype” as used herein is information regarding the presence or absence of one or more genetic markers in a given chromosomal region in a subject. A haplotype can consist of a variety of genetic markers, including indels (insertions or deletions of the DNA at particular locations on the chromosome); single nucleotide polymorphisms (SNPs) in which a particular nucleotide is changed; microsatellites; and minisatellites.

“Linkage disequilibrium” refers to when the observed frequencies of haplotypes in a population does not agree with haplotype frequencies predicted by multiplying together the frequency of individual genetic markers in each haplotype.

The term “chromosome” as used herein refers to a gene carrier of a cell that is derived from chromatin and comprises DNA and protein components (e.g., histones). The conventional internationally recognized individual human genome chromosome numbering identification system is employed herein. The size of an individual chromosome can vary from one type to another with a given multi-chromosomal genome and from one genome to another. In the case of the human genome, the entire DNA mass of a given chromosome is usually greater than about 100,000,000 base pairs. For example, the size of the entire human genome is about 3×10⁹ base pairs.

The term “gene” refers to a DNA sequence in a chromosome that includes a sequence that codes for a product (either RNA or its translation product, a polypeptide). A gene contains a coding region and includes regions preceding and following the coding region (termed respectively “leader” and “trailer”). The coding region is comprised of a plurality of coding segments (“exons”) and intervening sequences (“introns”) between individual coding segments.

The term “probe” refers to an oligonucleotide. A probe can be single stranded at the time of hybridization to a target. As used herein, probes include primers, i.e., oligonucleotides that can be used to prime a reaction, e.g., a PCR reaction.

The term “label” or “label containing moiety” refers in a moiety capable of detection, such as a radioactive isotope or group containing same, and nonisotopic labels, such as enzymes, biotin, avidin, streptavidin, digoxygenin, luminescent agents, dyes, haptens, and the like. Luminescent agents, depending upon the source of exciting energy, can be classified as radioluminescent, chemiluminescent, bioluminescent, and photoluminescent (including fluorescent and phosphorescent). A probe described herein can be bound, e.g., chemically bound to label-containing moieties or can be suitable to be so bound. The probe can be directly or indirectly labeled.

The term “direct label probe” (or “directly labeled probe”) refers to a nucleic acid probe whose label after hybrid formation with a target is detectable without further reactive processing of hybrid. The term “indirect label probe” (or “indirectly labeled probe”) refers to a nucleic acid probe whose label after hybrid formation with a target is further reacted in subsequent processing with one or more reagents to associate therewith one or more moieties that finally result in a detectable entity.

The terms “target,” “DNA target,” or “DNA target region” refers to a nucleotide sequence that occurs at a specific chromosomal location. Each such sequence or portion is preferably at least partially, single stranded (e.g., denatured) at the time of hybridization. When the target nucleotide sequences are located only in a single region or fraction of a given chromosome, the term “target region” is sometimes used. Targets for hybridization can be derived from specimens which include, but are not limited to, chromosomes or regions of chromosomes in normal, diseased or malignant human cells, either interphase or at any state of meiosis or mitosis, and either extracted or derived from living or postmortem tissues, organs or fluids; germinal cells including sperm and egg cells, or cells from zygotes, fetuses, or embryos, or chorionic or amniotic cells, or cells from any other germinating body; cells grown in vitro, from either long-term or short-term culture, and either normal, immortalized or transformed; inter- or intraspecific hybrids of different types of cells or differentiation states of these cells; individual chromosomes or portions of chromosomes, or translocated, deleted or other damaged chromosomes, isolated by any of a number of means known to those with skill in the art, including libraries of such chromosomes cloned and propagated in prokaryotic or other cloning vectors, or amplified in vitro by means well known to those with skill; or any forensic material, including but not limited to blood, or other samples.

The term “hybrid” refers to the product of a hybridization procedure between a probe and a target.

The term “hybridizing conditions” has general reference to the combinations of conditions that are employable in a given hybridization procedure to produce hybrids, such conditions typically involving controlled temperature, liquid phase, and contact between a probe (or probe composition) and a target. Conveniently and preferably, at least one denaturation step precedes a step wherein a probe or probe composition is contacted with a target. Guidance for performing hybridization reactions can be found in Ausubel et al., Current Protocols in Molecular Biology, John Wiley & Sons, N.Y. (2003), 6.3.1-6.3.6. Aqueous and nonaqueous methods are described in that reference and either can be used. Hybridization conditions referred to herein are a 50% formamide, 2×SSC wash for 10 minutes at 45° C. followed by a 2×SSC wash for 10 minutes at 37° C.

Calculations of “identity” between two sequences can be performed as follows. The sequences are aligned for optimal comparison purposes (e.g., gaps can be introduced in one or both of a first and a second nucleic acid sequence for optimal alignment and non-identical sequences can be disregarded for comparison purposes). The length of a sequence aligned for comparison purposes is at least 50% (e.g., at least 60%, 70%, 80%, 90% or 100%) of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In some embodiments, the percent identity between two nucleotide sequences is determined using the GAP program in the GCG software package, using a Blossum 62 scoring matrix with a gap penalty of 12, a gap extend penalty of 4, and a frameshift gap penalty of 5.

The term “nonspecific binding DNA” refers to DNA which is complementary to DNA segments of a probe, which DNA occurs in at least one other position in a genome, outside of a selected chromosomal target region within that genome. An example of nonspecific binding DNA comprises a class of DNA repeated segments whose members commonly occur in more than one chromosome or chromosome region. Such common repetitive segments tend to hybridize to a greater extent than other DNA segments that are present in probe composition.

Samples that are suitable for use in the methods described herein contain genetic material, e.g., genomic DNA (gDNA). Genomic DNA is typically extracted from biological samples such as blood or mucosal scrapings of the lining of the mouth, but can be extracted from other biological samples including urine or expectorant. The sample itself will typically consist of nucleated cells (e.g., blood or buccal cells) or tissue removed from the subject. The subject can be an adult, child, fetus, or embryo. In some embodiments, the sample is obtained prenatally, either from a fetus or embryo or from the mother (e.g., from fetal or embryonic cells in the maternal circulation). Methods and reagents are known in the art for obtaining, processing, and analyzing samples. In some embodiments, the sample is obtained with the assistance of a health care provider, e.g., to draw blood. In some embodiments, the sample is obtained without the assistance of a health care provider, e.g., where the sample is obtained non-invasively, such as a sample comprising buccal cells that is obtained using a buccal swab or brush, or a mouthwash sample.

In some cases, a biological sample may be processed for DNA isolation. For example, DNA in a cell or tissue sample can be separated from other components of the sample. Cells can be harvested from a biological sample using standard techniques known in the art. For example, cells can be harvested by centrifuging a cell sample and resuspending the pelleted cells. The cells can be resuspended in a buffered solution such as phosphate-buffered saline (PBS). After centrifuging the cell suspension to obtain a cell pellet, the cells can be lysed to extract DNA, e.g., gDNA. See, e.g., Ausubel et al., Current Protocols in Molecular Biology, John Wiley & Sons, N.Y. (2003). The sample can be concentrated and/or purified to isolate DNA. All samples obtained from a subject, including those subjected to any sort of further processing, are considered to be obtained from the subject. Routine methods can be used to extract genomic DNA from a biological sample, including, for example, phenol extraction. Alternatively, genomic DNA can be extracted with kits such as the QIAamp® Tissue Kit (Qiagen, Chatsworth, Calif.) and the Wizard® Genomic DNA purification kit (Promega). Non-limiting examples of sources of samples include urine, blood, and tissue.

The absence or presence of an allele or genotype associated with AMD as described herein can be determined using methods known in the art. For example, gel electrophoresis, capillary electrophoresis, size exclusion chromatography, sequencing, and/or arrays can be used to detect the presence or absence of the allele or genotype. Amplification of nucleic acids, where desirable, can be accomplished using methods known in the art, e.g., PCR. In one example, a sample (e.g., a sample comprising genomic DNA), is obtained from a subject. The DNA in the sample is then examined to identify or detect the presence of an allele or genotype as described herein. The allele or genotype can be identified or determined by any method described herein, e.g., by sequencing or by hybridization of the gene in the genomic DNA, RNA, or cDNA to a nucleic acid probe, e.g., a DNA probe (which includes cDNA and oligonucleotide probes) or an RNA probe. The nucleic acid probe can be designed to specifically or preferentially hybridize with a particular polymorphic variant.

Other methods of nucleic acid analysis can include direct manual sequencing (Church and Gilbert, Proc. Natl. Acad. Sci. USA 81:1991-1995 (1988); Sanger et al., Proc. Natl. Acad. Sci. USA 74:5463-5467 (1977); Beavis et al., U.S. Pat. No. 5,288,644); automated fluorescent sequencing; single-stranded conformation polymorphism assays (SSCP) (Schafer et al., Nat. Biotechnol. 15:33-39 (1995)); clamped denaturing gel electrophoresis (CDGE); two-dimensional gel electrophoresis (2DGE or TDGE); conformational sensitive gel electrophoresis (CSGE); denaturing gradient gel electrophoresis (DGGE) (Sheffield et al., Proc. Natl. Acad. Sci. USA 86:232-236 (1989)); denaturing high performance liquid chromatography (DHPLC, Underhill et al., Genome Res. 7:996-1005 (1997)); infrared matrix-assisted laser desorption/ionization (IR-MALDI) mass spectrometry (WO 99/57318); mobility shift analysis (Orita et al., Proc. Natl. Acad. Sci. USA 86:2766-2770 (1989)); restriction enzyme analysis (Flavell et al., Cell 15:25 (1978); Geever et al., Proc. Natl. Acad. Sci. USA 78:5081 (1981)); quantitative real-time PCR (Raca et al., Genet Test 8(4):387-94 (2004)); heteroduplex analysis; chemical mismatch cleavage (CMC) (Cotton et al., Proc. Natl. Acad. Sci. USA 85:4397-4401 (1985)); RNase protection assays (Myers et al., Science 230:1242 (1985)); use of polypeptides that recognize nucleotide mismatches, e.g., E. coli mutS protein; allele-specific PCR, and combinations of such methods. See, e.g., Gerber et al., U.S. Patent Publication No. 2004/0014095, which is incorporated herein by reference in its entirety.

Sequence analysis can also be used to detect specific polymorphic variants. For example, polymorphic variants can be detected by sequencing exons, introns, 5′ untranslated sequences, or 3′ untranslated sequences. A sample comprising DNA or RNA is obtained from the subject. PCR or other appropriate methods can be used to amplify a portion encompassing the polymorphic site, if desired. The sequence is then ascertained, using any standard method, and the presence of a polymorphic variant is determined. Real-time pyrophosphate DNA sequencing is yet another approach to detection of polymorphisms and polymorphic variants (Alderborn et al., Genome Research 10(8):1249-1258 (2000)). Additional methods include, for example, PCR amplification in combination with denaturing high performance liquid chromatography (dHPLC) (Underhill et al., Genome Research 7(10):996-1005 (1997)).

In order to detect polymorphisms and/or polymorphic variants, it will frequently be desirable to amplify a portion of genomic DNA (gDNA) encompassing the polymorphic site. Such regions can be amplified and isolated by PCR using oligonucleotide primers designed based on genomic and/or cDNA sequences that flank the site. PCR refers to procedures in which target nucleic acid (e.g., genomic DNA) is amplified in a manner similar to that described in U.S. Pat. No. 4,683,195, and subsequent modifications of the procedure described therein. Generally, sequence information from the ends of the region of interest or beyond are used to design oligonucleotide primers that are identical or similar in sequence to opposite strands of a potential template to be amplified. See e.g., PCR Primer: A Laboratory Manual, Dieffenbach and Dveksler, (Eds.); McPherson et al., PCR Basics: From Background to Bench (Springer Verlag, 2000); Mattila et al., Nucleic Acids Res., 19:4967 (1991); Eckert et al., PCR Methods and Applications, 1:17 (1991); PCR (eds. McPherson et al., IRL Press, Oxford); and U.S. Pat. No. 4,683,202. Other amplification methods that may be employed include the ligase chain reaction (LCR) (Wu and Wallace, Genomics 4:560 (1989), Landegren et al., Science 241:1077 (1988), transcription amplification (Kwoh et al., Proc. Natl. Acad. Sci. USA 86:1173 (1989)), self-sustained sequence replication (Guatelli et al., Proc. Nat. Acad. Sci. USA 87:1874 (1990)), and nucleic acid based sequence amplification (NASBA). Guidelines for selecting primers for PCR amplification are well known in the art. See, e.g., McPherson et al., PCR Basics: From Background to Bench, Springer-Verlag, 2000. A variety of computer programs for designing primers are available, e.g., ‘Oligo’ (National Biosciences, Inc, Plymouth Minn.), MacVector (Kodak/IBI), and the GCG suite of sequence analysis programs (Genetics Computer Group, Madison, Wis. 53711).

In some cases, PCR conditions and primers can be developed that amplify a product only when the variant allele is present or only when the wild type allele is present (MSPCR or allele-specific PCR). For example, patient DNA and a control can be amplified separately using either a wild type primer or a primer specific for the variant allele. Each set of reactions is then examined for the presence of amplification products using standard methods to visualize the DNA. For example, the reactions can be electrophoresed through an agarose gel and the DNA visualized by staining with ethidium bromide or other DNA intercalating dye. In DNA samples from heterozygous patients, reaction products would be detected in each reaction.

In some embodiments, a peptide nucleic acid (PNA) probe can be used instead of a nucleic acid probe in the hybridization methods described above. PNA is a DNA mimetic with a peptide-like, inorganic backbone, e.g., N-(2-aminoethyl)glycine units, with an organic base (A, G, C, T or U) attached to the glycine nitrogen via a methylene carbonyl linker (see, e.g., Nielsen et al., Bioconjugate Chemistry, The American Chemical Society, 5:1 (1994)). The PNA probe can be designed to specifically hybridize to a nucleic acid comprising a polymorphic variant conferring susceptibility to or indicative of the presence of AMD.

In some cases, allele-specific oligonucleotides can also be used to detect the presence of a polymorphic variant. For example, polymorphic variants can be detected by performing allele-specific hybridization or allele-specific restriction digests. Allele specific hybridization is an example of a method that can be used to detect sequence variants, including complete genotypes of a subject (e.g., a mammal such as a human). See Stoneking et al., Am. J. Hum. Genet. 48:370-382 (1991); and Prince et al., Genome Res. 11:152-162 (2001). An “allele-specific oligonucleotide” (also referred to herein as an “allele-specific oligonucleotide probe”) is an oligonucleotide that is specific for particular a polymorphism can be prepared using standard methods (see Ausubel et al., Current Protocols in Molecular Biology, supra). Allele-specific oligonucleotide probes typically can be approximately 10-50 base pairs, preferably approximately 15-30 base pairs, that specifically hybridizes to a nucleic acid region that contains a polymorphism. Hybridization conditions are selected such that a nucleic acid probe can specifically bind to the sequence of interest, e.g., the variant nucleic acid sequence. Such hybridizations typically are performed under high stringency as some sequence variants include only a single nucleotide difference. In some cases, dot-blot hybridization of amplified oligonucleotides with allele-specific oligonucleotide (ASO) probes can be performed. See, for example, Saiki et al., Nature (London) 324:163-166 (1986).

In some embodiments, allele-specific restriction digest analysis can be used to detect the existence of a polymorphic variant of a polymorphism, if alternate polymorphic variants of the polymorphism result in the creation or elimination of a restriction site. Allele-specific restriction digests can be performed in the following manner. A sample containing genomic DNA is obtained from the individual and genomic DNA is isolated for analysis. For nucleotide sequence variants that introduce a restriction site, restriction digest with the particular restriction enzyme can differentiate the alleles. In some cases, polymerase chain reaction (PCR) can be used to amplify a region comprising the polymorphic site, and restriction fragment length polymorphism analysis is conducted (see Ausubel et al., Current Protocols in Molecular Biology, supra). The digestion pattern of the relevant DNA fragment indicates the presence or absence of a particular polymorphic variant of the polymorphism and is therefore indicative of the presence or absence of susceptibility to AMD. For sequence variants that do not alter a common restriction site, mutagenic primers can be designed that introduce a restriction site when the variant allele is present or when the wild type allele is present. For example, a portion of a nucleic acid can be amplified using the mutagenic primer and a wild type primer, followed by digest with the appropriate restriction endonuclease.

In some embodiments, fluorescence polarization template-directed dye-terminator incorporation (FP-TDI) is used to determine which of multiple polymorphic variants of a polymorphism is present in a subject (Chen et al., Genome Research 9(5):492-498 (1999)). Rather than involving use of allele-specific probes or primers, this method employs primers that terminate adjacent to a polymorphic site, so that extension of the primer by a single nucleotide results in incorporation of a nucleotide complementary to the polymorphic variant at the polymorphic site.

In some cases, DNA containing an amplified portion may be dot-blotted, using standard methods (see Ausubel et al., Current Protocols in Molecular Biology, supra), and the blot contacted with the oligonucleotide probe. The presence of specific hybridization of the probe to the DNA is then detected. Specific hybridization of an allele-specific oligonucleotide probe (specific for a polymorphic variant indicative of susceptibility to AMD) to DNA from the subject is indicative of susceptibility to AMD.

The methods can include determining the genotype of a subject with respect to both copies of the polymorphic site present in the genome. For example, the complete genotype may be characterized as −/−, as −/+, or as +/+, where a minus sign indicates the presence of the reference or wild type sequence at the polymorphic site, and the plus sign indicates the presence of a polymorphic variant other than the reference sequence. If multiple polymorphic variants exist at a site, this can be appropriately indicated by specifying which ones are present in the subject. Any of the detection means described herein can be used to determine the genotype of a subject with respect to one or both copies of the polymorphism present in the subject's genome.

Methods of nucleic acid analysis to detect polymorphisms and/or polymorphic variants can include, e.g., microarray analysis. Hybridization methods, such as Southern analysis, Northern analysis, or in situ hybridizations, can also be used (see Ausubel et al., Current Protocols in Molecular Biology, eds., John Wiley & Sons (2003)). To detect microdeletions, fluorescence in situ hybridization (FISH) using DNA probes that are directed to a putatively deleted region in a chromosome can be used. For example, probes that detect all or a part of a microsatellite marker can be used to detect microdeletions in the region that contains that marker.

In some embodiments, it is desirable to employ methods that can detect the presence of multiple polymorphisms (e.g., polymorphic variants at a plurality of polymorphic sites) in parallel or substantially simultaneously. Oligonucleotide arrays represent one suitable means for doing so. Other methods, including methods in which reactions (e.g., amplification, hybridization) are performed in individual vessels, e.g., within individual wells of a multi-well plate or other vessel may also be performed so as to detect the presence of multiple polymorphic variants (e.g., polymorphic variants at a plurality of polymorphic sites) in parallel or substantially simultaneously according to certain embodiments.

Additional Genetic Markers of AMD

A number of additional genetic markers of AMD are known in the art, and the methods described herein can include determining the identity of additional alleles of polymorphisms associated with altered risk of AMD, e.g., to detect the presence of alleles associated with increased risk of AMD.

For example, variants in the complement factor H(CFH), complement component 3 (C3), ARMS2/HTRA1, and complement factor B (CFB) genes have been identified that are associated with risk of AMD (Swaroop et al., Annu Rev Genomics Hum Genet. 2009; 10:19-43). As one example, the methods can include detecting the presence of a Y402H variant of complement factor H(CFH) (a “C” allele at rs1061170). A number of additional variants are listed in FIG. 3. See, e.g., Gold et al., Nature Genetics, 2006; 38(4):458-462; Maller et al., Nature Genetics, 2006; 38(9):1055-1059; Yates et al., New England Journal of Medicine, 2007; 357(6):553-561; Gehrs et al., Arch Ophthalmol. 2010; 128(3):349-358; Kaur et al., Invest Ophthalmol V is Sci. 2008; 49:1771-1776; Kaur et al., Invest. Ophthalmol. Vis. Sci. 2010; 51(1):59-63; and Swaroop et al., Human Molecular Genetics, 2007; 16(2):R174-R182. These additional markers can be used in combination with the markers described herein to provide a risk (or protective) haplotype. For example, in some embodiments the methods described herein include determining the identity of alleles at one or both of rs1858993 and/or rs1061170, wherein the presence of a “G” allele at rs1858993 and/or a “C” allele at rs1061170 in a subject indicates that the subject has an increased risk of developing AMD, e.g., late stage AMD.

Risk Factors for AMD

A number of risk factors are known to be associated with an increased risk of development or progression of AMD. In some embodiments, the risk factor is family history of AMD, increasing age (e.g., above 60, 70, 80 or 90 years of age), sex (women have a higher risk), smoking history, obesity, e.g., high body mass index (BMI, e.g., a BMI of >25), high waist circumference, high waist-hip ratio, weight change since age 20, dietary fat intake, high linoleic acid intake, and/or elevated cholesterol levels. For information regarding AMD risk factors, diagnosis, and treatment, see Rosenfeld et al., “Age-Related Macular Degeneration” in Ophthalmology: Expert Consult, Yanoff and Duker, Eds., Mosby; 3 edition (2008), and Pavan-Langston, Manual of Ocular Diagnosis and Therapy, Lippincott Williams & Wilkins; Sixth edition (2007).

Communicating Risk Assessment

This document also provides methods and materials to assist medical or research professionals in determining whether or not a subject has or is at risk for developing AMD. Medical professionals can be, for example, doctors, nurses, medical laboratory technologists, and pharmacists. Research professionals can be, for example, principle investigators, research technicians, postdoctoral trainees, and graduate students. A professional can be assisted by (1) determining whether specific polymorphic variants are present in a biological sample from a subject, and (2) communicating information about polymorphic variants to that professional.

After information about specific polymorphic variants is reported, a medical professional can take one or more actions that can affect patient care. For example, a medical professional can record information in the patient's medical record regarding the diagnosis or risk of the patient to develop AMD. In some cases, a medical professional can record information regarding risk assessment, or otherwise transform the patient's medical record, to reflect the patient's current medical condition. In some cases, a medical professional can review and evaluate a patient's entire medical record and assess multiple treatment strategies for clinical intervention of a patient's condition.

A medical professional can initiate or modify treatment after receiving information regarding a patient's diagnosis of or risk of developing AMD, for example. In some cases, a medical professional can recommend a change in therapy. In some cases, a medical professional can enroll a patient in a clinical trial for, by way of example, detecting correlations between an allele or genotype as described herein and any measurable or quantifiable parameter relating to the outcome of the treatment as described above.

A medical professional can communicate information regarding a patient's diagnosis of or risk of developing AMD to a patient or a patient's family. In some cases, a medical professional can provide a patient and/or a patient's family with information regarding AMD diagnosis and risk assessment information, including treatment options, prognosis, and referrals to specialists. In some cases, a medical professional can provide a copy of a patient's medical records to a specialist.

A research professional can apply information regarding a subject's diagnosis of or risk of developing AMD to advance scientific research. For example, a researcher can compile data on specific polymorphic variants. In some cases, a research professional can obtain a subject's allele(s) or genotype as described herein to evaluate a subject's enrollment, or continued participation, in a research study or clinical trial. In some cases, a research professional can communicate information regarding a subject's diagnosis of or risk of developing AMD to a medical professional. In some cases, a research professional can refer a subject to a medical professional.

Any appropriate method can be used to communicate information to another person (e.g., a professional). For example, information can be given directly or indirectly to a professional. For example, a laboratory technician can input a patient's polymorphic variant allele(s) or genotype as described herein into a computer-based record. In some cases, information is communicated by making a physical alteration to medical or research records. For example, a medical professional can make a permanent notation or flag a medical record for communicating the risk assessment to other medical professionals reviewing the record. In addition, any type of communication can be used to communicate the risk assessment information. For example, mail, e-mail, telephone, and face-to-face interactions can be used. The information also can be communicated to a professional by making that information electronically available to the professional. For example, the information can be communicated to a professional by placing the information on a computer database such that the professional can access the information. In addition, the information can be communicated to a hospital, clinic, or research facility serving as an agent for the professional.

Methods of Treatment

In some embodiments, the methods described herein include administering to a subject a treatment for AMD. As used herein a “treatment” can be any treatment that is known or suspect to reduce risk of, prevent (but need not be 100% preventative), or delay the development of AMD. A number of such treatments are known in the art, e.g., photodynamic therapy or laser photocoagulation to treat wet AMD, diet and exercise regiments, and/or vitamin supplements, e.g., as described in AREDS Research Group, Arch Ophthalmol 2001:119, 1417-1436; Seddon et al, JAMA, 1994; 272: 1413-1420, and U.S. Pat. No. 6,660,297. For additional information regarding AMD diagnosis and treatment, see Rosenfeld et al., “Age-Related Macular Degeneration” in Ophthalmology: Expert Consult, Yanoff and Duker, Eds., Mosby; 3 edition (2008), and Pavan-Langston, Manual of Ocular Diagnosis and Therapy, Lippincott Williams & Wilkins; Sixth edition (2007).

In some embodiments, the methods include administering to the subject an anti-angiogenesis agent, e.g., anecortave acetate (Alcon), which reduces production of matrix metalloproteinase, a key agent in the growth of neovascular membranes; or an agent that inhibits vascular endothelial growth factor (VEGF). A number of inhibitors of VEGF signalling are known in the art and can include, e.g., ZD6474 (Tuccillo et al., Clin Cancer Res. 2005 Feb. 1; 11(3):1268-76); COX-2, Tie2 receptor, angiopoietin, and neuropilin inhibitors; pigment epithelium-derived factor (PEDF), endostatin, and angiostatin (21-25); VEGF inhibitory aptamers, e.g., Macugen™ (pegaptanib, Pfizer); antibodies or fragments thereof, e.g., anti-VEGF antibodies, e.g., bevacizumab (Avastin™, Genentech), or fragments thereof, e.g., ranibizumab (Lucentis™, Genetech); soluble fms-like tyrosine kinase 1 (sFlt1) polypeptides or polynucleotides (Harris et al., Clin Cancer Res. 2001 July; 7(7):1992-7; U.S. Pat. No. 5,861,484); PTK787/ZK222 584 (Maier et al., Graefes Arch Clin Exp Ophthalmol. 243(6):593-600 (2005); KRN633 (Maier et al., Mol Cancer Ther. 2004 December; 3(12):1639-49); VEGF-Trap™ (Regeneron); intravitreal steroids, e.g., triamcinolone; and Alpha2-antiplasmin (Matsuno et al, Blood 2003; 120:3621-3628). For reviews of VEGF and its inhibitors, see, e.g., Campochiaro, Ocular neovascularisation and excessive vascular permeability, Expert Opin Biol Ther. 2004 September; 4(9):1395-402; Ferrara, Vascular Endothelial Growth Factor: Basic Science and Clinical Progress, Endocr. Rev., Aug. 1, 2004; 25(4):581-611; and Verheul and Pinedo, Vascular endothelial growth factor and its inhibitors, Drugs Today (Barc). 2003; 39 Suppl C:81-93.

Finally, triamcinolone acetate is approved for the treatment of the neovascular stage of AMD, administered by an intravitreal injection.

A number of diet and exercise modifications can be administered to reduce the risk of development of AMD or progression to advanced AMD. For example, decreased fat intake and increased intake of antioxidants, fish, and nuts have been shown to be related to decreased risk of developing AMD. See, e.g., Seddon et al., Arch Ophthalmol. 2003 June; 121(6):785-92, and Seddon et al., Arch Ophthalmol. 2003 December; 121(12):1728-37. Erratum in: Arch Ophthalmol. 2004 March; 122(3):426. A number of nutritional and vitamin supplements are also available that are intended to enhance eye health, including lutein, xeathanthin, vitamin A (e.g., as beta-carotene or lutein), vitamins C and E, and minerals zinc and copper, see, e.g., Age-Related Eye Disease Study Research Group, Arch Ophthalmol. 2001; 119:1417-1436; Krishnadev et al., Curr Opin Ophthalmol. 2010; 21(3):184-9; and Johnson, Curr Opin Clin Nutr Metab Care. 2010 January; 13(1):28-33.

### EXAMPLES

The invention is further described in the following examples, which do not limit the scope of the invention described in the claims.

### Example 1

A positional candidate association analysis was performed within the Women's Genome Health Study (WGHS) (Ridker et al., Clin Chem. 2008; 54:249-255; Zee et al., Clin Chim Acta. 2011; 412:199-202) The WGHS is a prospective cohort of 28,346 initially healthy female health care professionals, 45 years or older at baseline, all with extensive clinical phenotype information and all with existing whole genome genotype information. The cohort derives from the subgroup of 28,346 or 71% of the Women's Health Study (WHS) who provided a baseline blood sample and consent for ongoing blood based analyses. The WHS was a randomized, placebo-controlled trial of aspirin and vitamin E in primary prevention of incident CVD and cancer with about 10 years of follow-up concluding in 2005 and now continuing in observational mode with a total of over 15 years of follow-up (Rexrode et al., J Womens Health Gend Based Med. 2000; 9:19-27). Much of the baseline clinical information derives from self-report, which has been proven highly accurate in this population due to the participants' occupations in health care. DNA was extracted from the baseline blood sample with non-Federal funding and used for genotyping with the Infinium-II assay on the Illumina (San Diego) HumanHap300 Duo “+” platform, containing a standard panel of about 317,000 SNPs designed to capture ˜70-80% of the common variation populations of European ancestry with LD r2>0.8 as well as additional SNPs (“+” content) selected by us either for suspected biological function or to increase coverage of genes known for involvement in CVD.

Quality control procedures for the genotype data in the WGHS enforced complete genotype information for at least 98% of the SNPs in each sample, and similarly at least 90% of the samples for each SNP, which further met Hardy Weinberg equilibrium with significance p-value >10⁻⁶. The multi-dimensional scaling procedure in PLINK was used to identify a subset of 23,294 samples with verified self-reported European ancestry. Within this sub-population, sub-European ancestry was estimated with EIGENSTRAT, allowing the potential to further correct for ancestry, although empirically these effects have been negligible for WGHS phenotypes with the exclusion of some biometric phenotypes, e.g. height (see, e.g., Price et al., Nat Genet. 38(8):904-9 (2006)).

Study participants for this investigation included the 23,294 women of confirmed European ancestry. Over 12 y of follow-up, 363 women developed AMD, including 161 with AMD associated with a visual acuity reduction to 20/30 or worse over, and 31 with neovascular AMD, the most visually devastating form of the disease.

A validated two-stage procedure was used to document incident cases of AMD (Seddon et al., JAMA. 276(14):1141-1146 (1996); Cho et al., Am J Clin Nutr. 73(2):209-218. (2001)). Participants were asked on each biennial study questionnaire about the diagnosis of AMD. When AMD was reported, permission to review medical records was requested. If permission was granted, a letter was sent to the participant's eye doctor to obtain information on the date of AMD diagnosis, best-corrected visual acuity at the most recent exam, and the chorio-retinal lesions present (drusen; RPE changes including atrophy, hypertrophy and RPE detachment; geographic atrophy; subretinal neovascular membrane; disciform scar), and other information. Cases were classified as neovascular AMD if there was an RPE detachment, subretinal neovascular membrane, or disciform scar not due to other causes (e.g. histoplasmosis, choroidal rupture). Participants were classified based on the most severely affected eye.

This analysis focused on SNPs covering approximately 5% of the genome within 2p25.1-2p16.2, 3p25.3-3p14.1, and 16p13-16q23.1, regions identified as the strongest candidates (apart from the regions on chromosomes 1 and 10 for which AMD-associated SNPs have already been identified) by a prior meta-analysis of genome-wide linkage studies (Fisher et al., Hum Mol Genet. 2005; 14:2257-2264).

For each SNP tested in each region, 1) the allele-frequency chi-square P-value along with the corresponding odds ratio (OR) and 95% confidence interval (CI), and 2) the OR (CI) and associated P-value for each SNP from an age-adjusted logistic regression model assuming an additive genetic model on the log-odds scale, was calculated. For each region separately, the p-value of the best SNP was then corrected for the number of SNPs in the region using the Sidak procedure as: corrected p-value=1-(1-uncorrected p-value)^{N SNPs}.

Results:

Women with visually significant AMD were older at baseline, and more likely to be current cigarette smokers, and to be obese (Table 1).

TABLE 1
Baseline characteristics of the study population

| | No AMD | AMD w/ VA loss |
|---|---|---|
| | (N = 22,657) | (N = 161) |
| Age, y | 54.5 | 65.1 |
| Current Cigarette | 12% | 17% |
| Smoking | | |
| Obesity | 17% | 22% |

Prior to testing the candidate regions, it was examined whether there was evidence of associations for AMD in the CFH and ARMS2 regions, and identified significant associations in both regions (Table 2).

TABLE 2
P-values for tests of association with AMD for established
loci in the WGHS

| | | | Neovascular |
|---|---|---|---|
| | All AMD | AMD w/ VA loss | AMD |
| | (N = 383) | (N = 161) | (N = 31) |
| CFH region | | | |
| Best SNP in region | 1.6 × 10⁻¹¹ | 8.8 × 10⁻¹⁴ | — |
| | rs10801555 | rs6685931 | |
| CFH Y402H* | 1.24 × 10⁻⁰⁹ | 7.37 × 10⁻¹⁰ | 0.048 |
| ARMS2 region | | | |
| Best SNP in region | | 3.95 × 10⁻⁰⁵ | 3.8 × 10⁻⁰⁶ |
| | | rs932275 | rs1566866 |
| ARMS2 A69S* | 1.48 × 10⁻⁰⁵ | 2.42 × 10⁻⁰⁵ | 0.0029 |

*SNP imputed from HapMap r22 using MACH v. 1.0.6 (Li et al., Genet Epidemiol. 34(8): 816-34 (2010)).

For the outcome of AMD with vision loss, SNP rs6685931 was the most statistically significant SNP identified in the CFH region (P=8.8×10⁻¹⁴), and SNP rs932275 was the most significant in the ARMS2 region (P=3.95×10⁻⁵). Values for SNPs CFHY402H and ARMS2 were imputed as these were not genotyped directly on the WGHS platform. Tests of these SNPs for association with AMD yielded p-values of 7.37×10⁻¹⁰ for CFH Y402H and 2.42×10⁻⁵ for ARMS2 A69S.

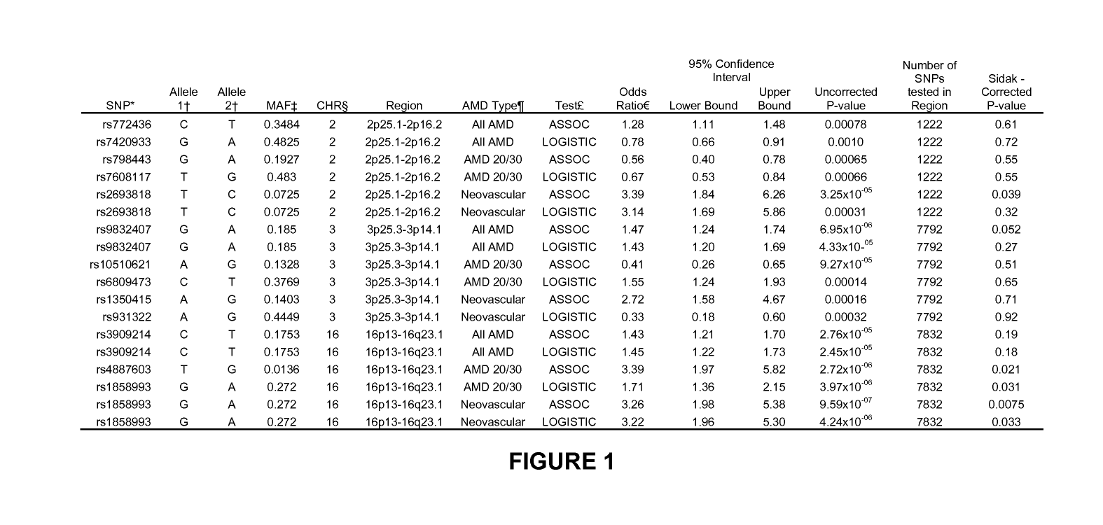

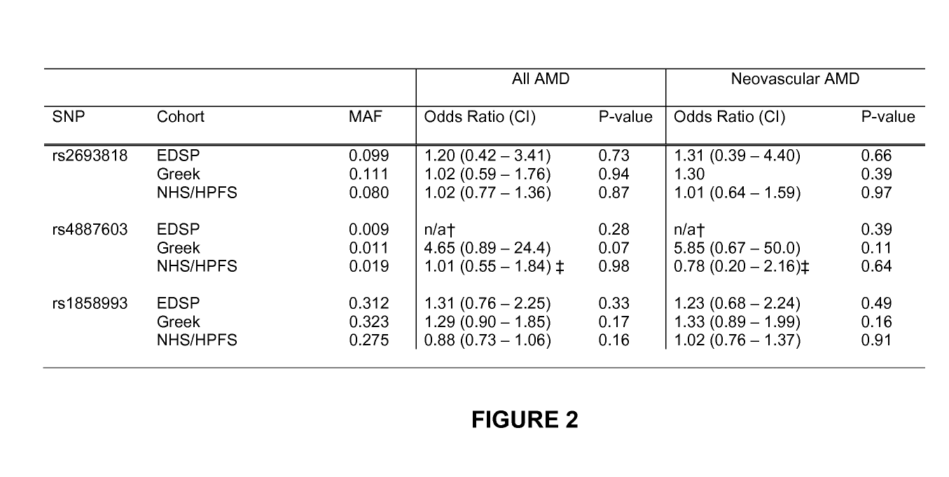

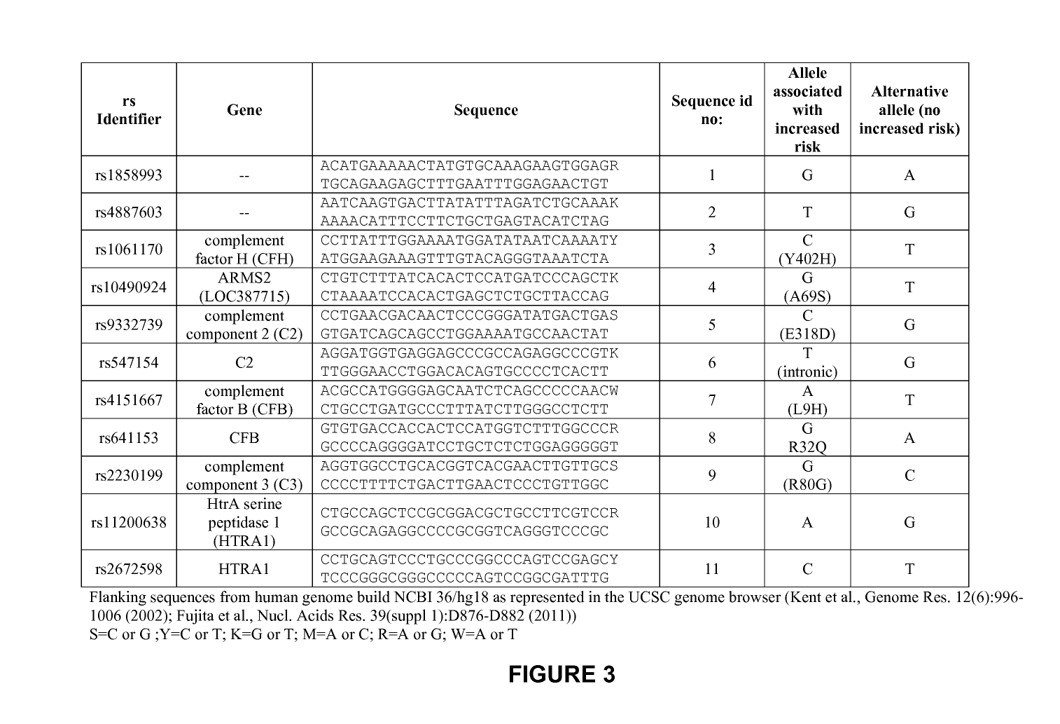

In analysis of the 3 candidate regions, two SNPs were identified on chromosome 16 that were significantly associated with the development of incident visually significant AMD, when adjusted for the number of SNPs evaluated in this region. These SNPs were: rs4887603 (OR=3.39, corrected P=0.02), and rs1858993 (OR=1.71, corrected P=0.01). SNP rs1858993 on chromosome 16 was also significantly associated with the development of the neovascular form of AMD, which occurred in 31 women (OR=3.25, corrected P=0.007), and this association remained significant after adjusting for all SNPs in all evaluated regions. One regionally significant SNP, rs2693818, was also identified on chromosome 2 associated with neovascular AMD (OR=3.39, corrected P=0.04). The detailed results are presented in FIG. 1.

### Example 2

SNPs identified as significant in the WGHS were further evaluated within three independent study populations: 1) a group of 457 incident cases of AMD and 1071 matched controls from the Nurses' Health Study (NHS) and Health Professionals Follow-up Study (HPFS) cohorts (Schaumberg et al., Arch Ophthalmol. 2007; 125:55-62, Schaumberg et al., 2010; Arch Ophthalmol. 128:1462-1471), 2) an extremely discordant sib-pair cohort of 150 individuals with AMD and 150 sibs without AMD (DeAngelis et al., Arch Ophthalmol. 2004; 122:575-580; Silveira et al., Vision Res. 2010; 50(7):698-715), and 3) a clinic-based AMD case-control study if 139 cases of AMD and 121 control subjects all from Greece (Silveira et al., Vision Res. 2010; 50(7):698-715). Association with AMD was tested in each population, and evaluated potential interactions on the multiplicative scale with the CFH Y402H and ARMS2 A69S polymorphisms. SNPs were genotyped in these populations using known methods.

Results:

In models adjusting for age, sex, and cigarette smoking, no statistically significant associations were observed in any of the three replication populations for all AMD cases and controls, or subgroups of neovascular AMD cases and controls (FIG. 2). Odds ratio estimates assuming an additive genetic model for the association of rs1858993 with all AMD cases were: 1.31 among the extremely discordant sib-pairs, 1.29 among the cases and controls from Greece, and 0.88 among incident cases and controls from the NHS/HPFS. Odds ratios for rs1858993 and neovascular AMD were: 1.23 among the extremely discordant sib-pairs, 1.33 among the cases and controls from Greece, and 1.02 for incident cases and controls from the NHS/HPFS. The age-, sex-, and smoking-adjusted pooled odds ratio estimates combining data from the three replication cohorts were 1.02 (P=0.79) and 1.14 (P=0.10) for all AMD cases and neovascular AMD cases, respectively.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of predicting risk of developing age-related macular degeneration (AMD) in a human subject, the method comprising:
obtaining a sample comprising DNA from the subject;
detecting in the sample one or both of:
(i) the presence of a “G” allele at polymorphism rs1858993, or
(ii) the presence of a “T” allele at polymorphism rs4887603; and
wherein the presence of a “G” allele at rs1858993, or a “T” allele at rs4887603 indicates that the subject has an increased risk of developing AMD.

2. The method of claim 1, further comprising selecting a subject who has an increased risk of developing age-related macular degeneration (AMD), based on the presence of one or both of a “G” allele at polymorphism rs1858993, or a “T” allele at polymorphism rs4887603; and selecting a regimen comprising a treatment for AMD for the subject.

3. The method of claim 1, further comprising detecting the presence of one or more additional genetic variants associated with altered risk of AMD.

4. The method of claim 3, wherein the additional genetic variants are selected from the group consisting of a Y402H variant of complement factor H(CFH), identified by the presence of a “C” allele at rs1061170; an A69S variant of ARMS2, identified by the presence of a “G” allele at rs10490924; an E318D variant of complement component 2 (C2), identified by the presence of a “C” allele at rs9332739; an L9H variant of complement factor B (CFB), identified by the presence of an “A” allele at rs4151667; an R32Q variant of complement factor B (CFB), identified by the presence of a “G” allele at rs641153; an R80G variant of complement component 3 (C3), identified by the presence of a “G” allele at rs2230199; an “A” allele at rs11200638; a “C” allele at rs2672598; and a “T” allele at rs547154.

5. The method of claim 1, wherein the AMD is neovascular AMD.

6. The method of claim 1, wherein detecting the presence of an allele comprises determining identity of the nucleotide at the polymorphism.

7. The method of claim 6, wherein determining the identity of the nucleotide comprises contacting the sample with a probe specific for a selected allele of the polymorphism, and detecting the formation of complexes between the probe and the selected allele of the polymorphism, wherein the formation of complexes between the probe and the test marker indicates the presence of the selected allele in the sample.

8. The method of claim 6, wherein determining the identity of an allele comprises determining the identity of the nucleotide at position 31 of one of SEQ ID NOs: 1 or 2.

9. The method of claim 1, wherein the subject is a patient having or suspected of having AMD.

10. The method of claim 1, wherein the subject has one or more risk factors associated with AMD.

11. The method of claim 1, further comprising selecting the subject based on the presence of one or more risk factors associated with AMD.

12. The method of claim 11, wherein the risk factors associated with AMD include one or more of: family history of AMD, age, sex, smoking history, obesity, body mass index (BMI), waist circumference, waist-hip ratio, weight change since age 20, dietary fat intake, linoleic acid intake, and elevated cholesterol levels.

13. The method of claim 1, wherein the subject has exhibited or exhibits symptoms of AMD.

14. The method of claim 1, further comprising selecting or excluding a subject for enrollment in a clinical trial based on the identity of the allele.

15. The method of claim 1, further comprising stratifying a subject population for analysis of a clinical trial based on the identity of the allele in the subjects.

16. The method of claim 1, further comprising monitoring the subject for the development of AMD.

17. The method of claim 2, further comprising administering the selected treatment to the subject.

18. The method of claim 1, comprising detecting in the sample the presence of a “G” allele at polymorphism rs1858993.

19. The method of claim 1, comprising detecting in the sample the presence of a “T” allele at polymorphism rs4887603.
